(19) United States
(12) Patent Application Publication
(10) Pub. No.: US 2024/0050699 A1
(43) Pub. Date: Feb. 15, 2024

(54) Catheter Handle

(71) Applicant: Tyche MedTech, Inc., Santa Clara, CA (US)

(72) Inventors: Anatoly Ryzhikh, Santa Clara, CA (US); Waseem Qureshi, Santa Clara, CA (US)

(73) Assignee: Tyche MedTech, Inc., Santa Clara, CA (US)

(21) Appl. No.: 17/818,744

(22) Filed: Aug. 10, 2022

(51) Int. Cl. A61M 25/01; A61M 25/09

(52) U.S. Cl. CPC A61M 25/0136; A61M 25/09; A61M 25/0147

(57) ABSTRACT

A control handle for a steerable catheter. A housing with a hollow interior region has a rotatable lever mechanism. Features are formed in the housing and on the lever mechanism for routing and affixing the catheter guidewire and to provide tension on the guidewire in a push/pull operation.

### TECHNICAL FIELD

This disclosure relates generally to catheters, and more specifically, to a control handle for a steerable catheter.

### BACKGROUND

A catheter is a well-known medical device consisting of a thin tube of medical grade material, such as a polyamide fabric, that is insertable into the body for introducing drugs or performing other surgical or analytical procedures. There are a wide variety of known catheter configurations tailored to specific medical applications, such as cardiovascular, urological, gastrointestinal, neurovascular, ophthalmic, etc. In one example, the catheter tube is configured with a number of electrodes (depending on the application) disposed at spaced-apart locations along the tube including a tip electrode at the distal end of the tube. Electrical wiring inside the catheter tube powers the electrodes. The catheter tube is coupled to a handle, and an electrical cable is coupled to the handle to provide a signal to the catheter wiring. Typical catheter tube diameters range from approximately 3-7 on the French scale (FR=3×mm measure).

Many medical applications benefit from a steerable catheter, where the handle includes a control mechanism for deflecting the distal end of the catheter tube in order to properly guide and position the catheter for the intended use. However, establishing and then fixing the position of the catheter tip can be problematic if the control mechanism is not effective at maintaining tip position, for example, due to slippage in the control mechanism.

Therefore, it would be desirable to provide a control mechanism for a steerable catheter that is simple in construction, easy to use, and reliably accurate in maintaining a deflected catheter position in use.

### BRIEF DESCRIPTION OF THE DRAWINGS

FIG. 1 is a perspective view of a catheter handle.

FIG. 2 is a perspective view of the bottom half of catheter handle of FIG. 1, with the top half removed to reveal a lever mechanism disposed in the interior of the handle.

FIG. 3 is a perspective view of the bottom half of the catheter handle of FIG. 2 without the lever mechanism.

FIG. 4 is a side plan sectional view through section 4-4 of the bottom half of the catheter handle of FIG. 3.

FIG. 5 is a perspective view of the top half of catheter handle of FIG. 1.

FIG. 6 is top plan view of the top half of the catheter handle of FIG. 5.

FIG. 7 is a top perspective view of the lever mechanism.

FIG. 8 is a side plan view of the lever mechanism of FIG. 7.

FIG. 9 is a perspective view of a spacer used with the lever mechanism of FIG. 7.

FIG. 10 is a perspective view of a threaded stud used with the lever mechanism of FIG. 7.

### DETAILED DESCRIPTION

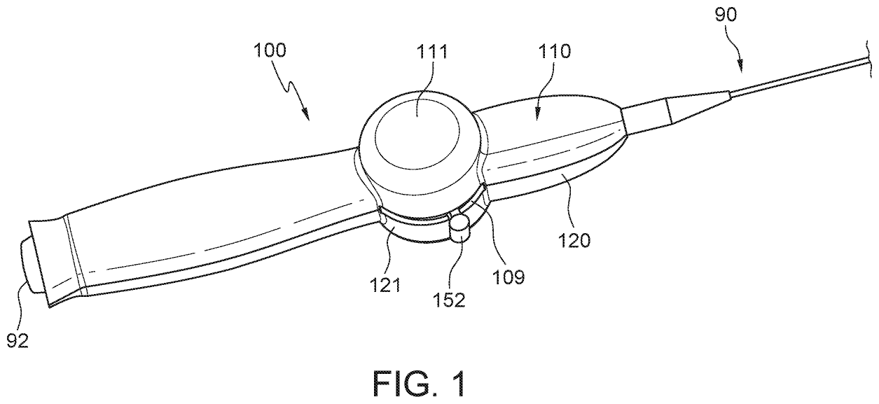

FIG. 1 illustrates one embodiment of a catheter control handle 100 for use with a flexible, steerable catheter 90 that is coupled to the front end of the handle. A connector 92 is affixed at the back end of the handle 100 for receiving and coupling an electrical cable that will transmit a signal to be applied to electrodes disposed along the catheter, via compatible wiring provided inside the handle (not shown).

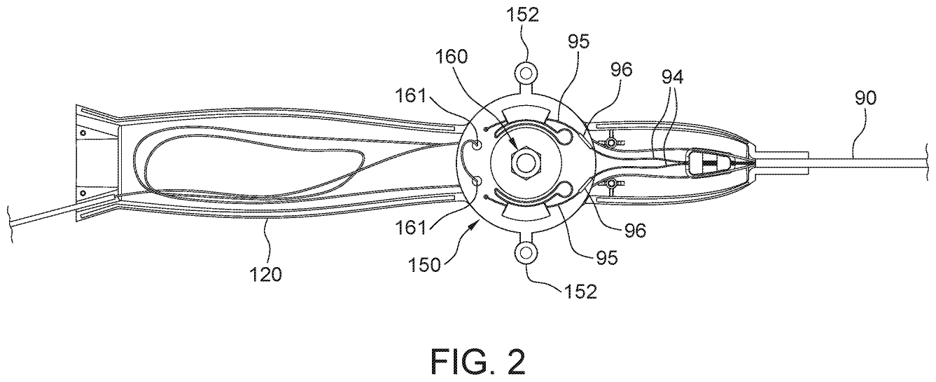

The catheter control handle 100 in this example is a two-part housing formed of molded polycarbonate and defined by a top part 110 coupled with a compatible bottom part 120. The top half 110 and bottom half 120 may be attached to each other, for example, by a suitable adhesive and/or plastic locking tabs or ridges in well-known manner, e.g., at or near the edges. A pair of circular portions 111, 121 are symmetrically formed in a central portion of the top and bottom parts 110, 120, respectively, for containing a lever mechanism 150 (see FIG. 2). The lever mechanism 150 is a substantially circular component that is rotatable within the circular portions 111, 121 by manually operating a lever arm 152 that extends from the lever mechanism through a slot 109 formed at the edge of each half of the handle. Operating the lever arm 152 directly rotates the lever mechanism 150 within the circular portions 111, 121 with the result that tip of the catheter is deflected. In this example embodiment, the range of operation of the lever mechanism (and hence deflection of the catheter tip) is approximately 90 degrees.

Referring now to FIG. 2, the bottom part 120 of handle 100 is shown, with lever mechanism 150 contained within the circular portion (not visible in this view) and mounted for rotation on a post assembly 160. The catheter tube 90 is affixed near the front end of the handle, and the center of the catheter guidewire 94 is fixed to the inside of the catheter tip at the distal end. The two legs of the guidewire extend from the proximate end of the catheter tube into the housing, are attached to a cable 95 at point 96, and the cable is wrapped around and affixed to the lever mechanism 150, as will be further described.

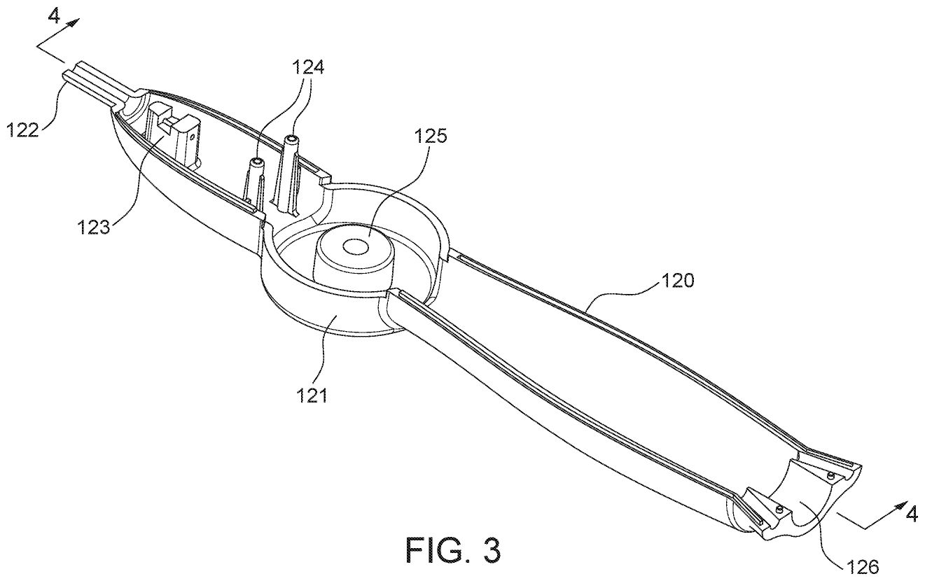

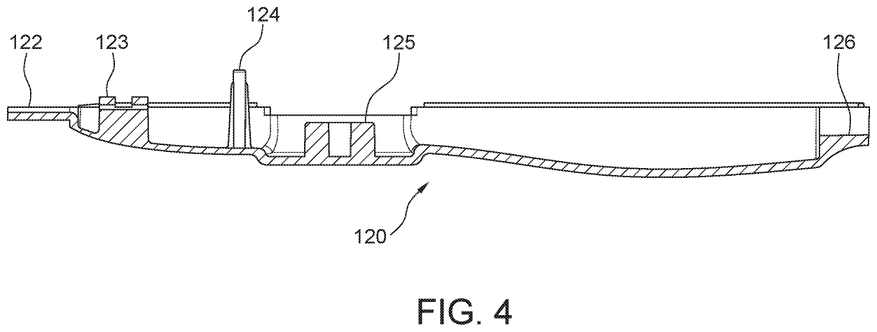

FIG. 3 illustrates the bottom part 120 without the lever mechanism 150, and FIG. 4 is a cross-sectional view. Advantageously, in this embodiment, the bottom part 120 includes features formed for (i) mounting the lever mechanism 150, and (ii) directing the guidewire 94 onto the lever mechanism. The bottom part 120 includes a half circle opening 122 at the front for receiving the catheter tube, and a saddle 123 formed as a first feature for securely holding the catheter tube in place at the front of the handle. A pair of posts 124 are symmetrically formed as second set of features and disposed and spaced apart between the saddle 123 and a circular plate 125, one near each opposing edge and ahead of the lever mechanism, to provide inward tension on each guidewire leg 94 before it wraps around and attaches to the lever mechanism. The circular plate 125 is formed in the circular portion 121 for mounting the lever mechanism. In this embodiment, the circular plate 125 is a hollow cylinder (toroid) into which the smooth portion 168 of the partially threaded stud 166 (see FIG. 10) is inserted and glued in a vertical orientation as part of the post assembly. A half circle opening 126 at the rear of the housing 120 is configured to receive a cable connector for the electrode signals.

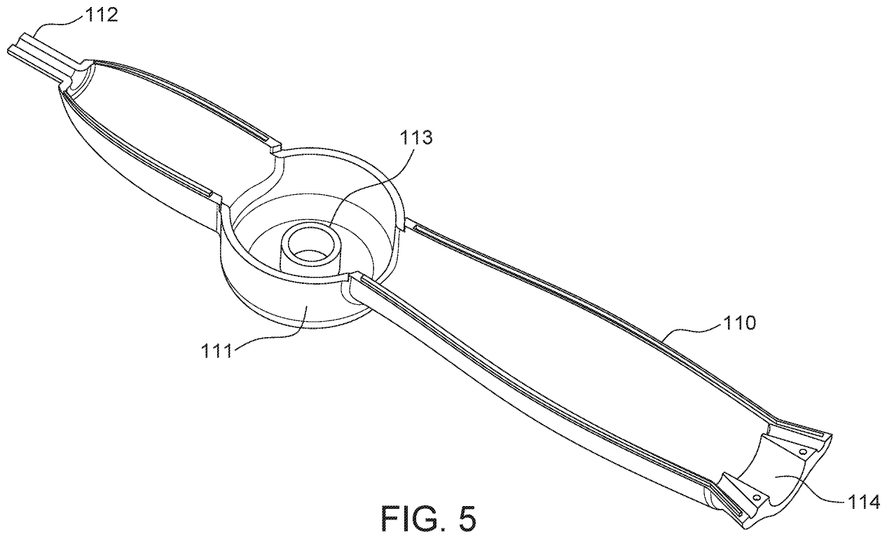

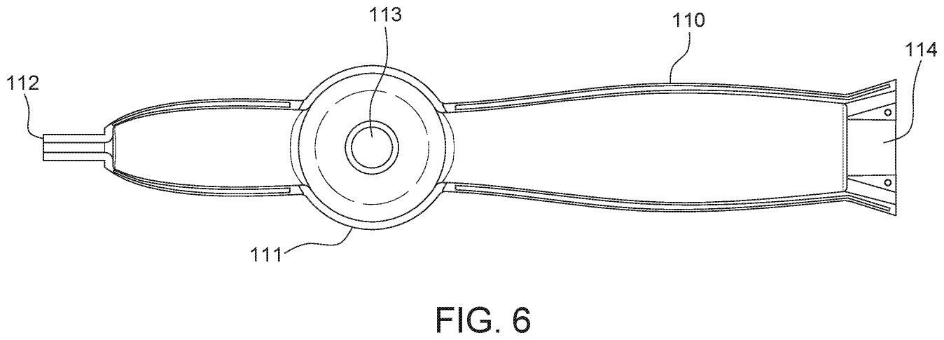

FIG. 5 illustrates the top part 110, and FIG. 6 is a top plan view of FIG. 5 (as illustrated). The top part 110 is compatible with the bottom part 120 and acts simply to close and seal the handle. A half circle opening 112 is formed at the front for the catheter tube. A dimple feature 113 is provided in the middle of the circular portion 111 as a receptacle and secure terminus for the post assembly. A half circle opening 114 at the rear is configured to receive a cable connector for the electrode signals.

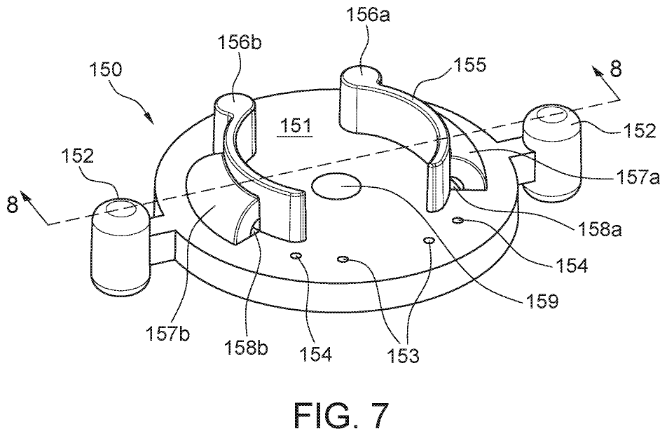

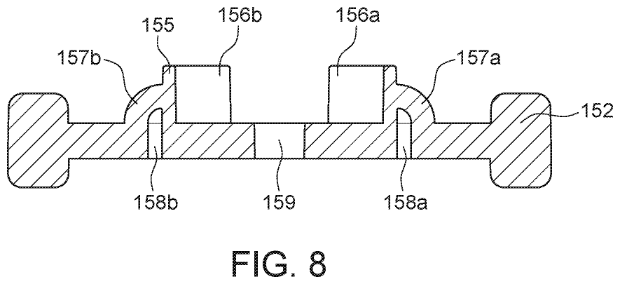

FIG. 7 illustrates the lever mechanism 150, and FIG. 8 is a cross-sectional view. The lever mechanism 150 is formed with a third set of features symmetrically disposed on top of a flat circular plate 125 for providing appropriate outward tension and travel to the guidewire in its push/pull operation to deflect the catheter tip. However, it should be noted that the guidewire 94 is advantageously soldered to a piece of cable 95, as shown in FIG. 2, and it is the cable that acts to push/pull the connected guidewire when the lever is rotated. The circular plate 151 has lever arms 152 extending from opposite sides of the plate, and when in a neutral position, the lever arms each extend at a right angle from the control handle. There are two pairs of holes 153, 154 formed at the rear of the plate 151.

A pair of semicircular walls 155 are vertically and symmetrically disposed on each side of the plate 151, with the same radius of curvature as the circular plate, each wall ending in a respective knob portion 156a, 156b. An open “lane” is defined between the walls 155 through the center of the plate 151, and in a rest or starting position, the open lane is oriented coaxially with the length of the housing, and thus the pair of semicircular walls are located to the left side and the right side in that orientation. The knob portions 156a, 156b are cylindrical with a much smaller radius of curvature than the wall 155 and circular plate 151. The knob portions 156a, 156b provide tension points for the legs of guidewire/cable as it travels in its push/pull operation. In combination with posts 124, which are positioned in the housing slightly outbound of the knob portions, the knob portions 156a, 156b act to provide additional tension and rotation range during push/pull operation. Attached to a central portion on the outside of each wall 155 are corresponding structures 157a, 157b that define respective tunnel portions 158a, 158b formed along each wall for routing the cable 95. Finally, a center hole 159 is formed for receiving the post assembly in order to mount the lever mechanism in the handle.

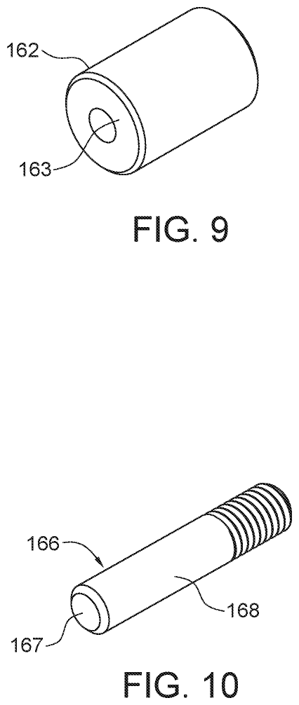

A piece of cable 95 is inserted into the tunnel 158a from the knob end 156a of the structure 157a, and is routed through the two pairs of holes 153 and 154 such that a loop is formed over the inner set of holes 153 on the top of the plate 151. The other end the cable 95 exits the tunnel 158b of the other structure 157b. During the cable insertion process, two spacers 162 (see FIG. 9) are inserted over the cable such that the spacers are seated over a respective one of the two inner holes 153.

Thus, the two ends of the guidewire 94 are soldered to the two ends of the cable 95 that is inserted all the way around the lever 151 from one knob 156a to the other knob 156b. The soldering of the guidewire ends to the respective cable ends is done midway between the posts 124 and the knobs 156 on either side of the lever 150. The guidewire runs on the inside of the post 124 in the handle and into the catheter tube. As previously noted, operating the lever arm 152 acts to rotate the lever mechanism thereby pulling one leg and pushing the other leg of the guidewire, with the result that the tip of the catheter is deflected in the “pulled” direction. The second set of features formed in the housing provide inward tension on the guidewire in operation while the third set of features formed on the lever mechanism provide outward tension

FIG. 9 illustrates a spacer 162 having an axial opening 163 through the spacer. The two spacers 162 sit on top of the lever plate 151 such that the axial opening 163 of each of the two spacers aligns with the two holes 153 respectively, on the lever plate. The threaded portion of the stud 166 (see FIG. 10) protrudes through the central hole 159 of the plate 151. The bottom portion of stud 166 is glued into the hole 125 formed in the bottom half of the handle 120. A nitrile washer as well as a steel washer is inserted over the threaded portion of the stud 166, and a locking nut is tightened over the thread 160, as shown in FIG. 2. The top threaded portion of the stud 166 is held by the dimple 113 formed in the top half 110 when the handle parts are sealed together.

The following is a description of approximate dimensions suitable for implementing the control handle as shown and described above. However, the dimensions are intended to exemplary only and not limiting.
handle overall length: 208.25 mm;
handle front opening (112, 122): 2.44 mm radius, over 15.00 mm tip region;
handle rear opening (114, 126): 13.40 mm radius, over 12.00 mm butt region;
handle circular portions (111, 121): 20.00 mm radius
table 125 in bottom circular portion (121): 17.00 mm radius centered in circular portion, with 5.35 mm radius center hole having female M5 threads;
spacer 162: made of 316 stainless steel, 22 mm radius with 5.25 mm radius center hole;
stud 166: made of 316 stainless steel, 5.00 mm radius, 25.00 mm length, M5×0.8 mm threads formed over bottom 8.00 mm of stud;

The overall length of the control handle 100 is approximately 208 mm. The front opening is approximately

While specific embodiments have been described by way of example, it should be understood that the invention is not limited to the disclosed embodiments. To the contrary, this disclosure is intended to cover various modifications and similar arrangements as would be apparent to those skilled in the art. Therefore, the scope of the appended claims should be accorded the broadest interpretation so as to encompass all such modifications and similar arrangements.

1. A control handle for a steerable catheter, comprising:
a housing having a front portion including an opening, the opening adapted for receiving a catheter tube having a guidewire affixed at both ends thereof inside a distal end of the catheter tube and a pair of guidewire legs extending from a proximate end of the catheter tube into the front portion of the housing;
a first feature formed proximate the opening in the front portion of the housing for affixing the catheter tube;
a lever assembly affixed within the housing;
a lever plate rotatably coupled with the lever assembly, the lever plate having at least one lever arm extending outside the housing and movable to rotate the lever plate on the lever assembly when operated, the lever plate having a pair of cable terminal points disposed symmetrically on a rear portion of the lever plate for connecting the pair of guidewire legs;
a plurality of second features disposed symmetrically within the front portion of the housing adjacent the lever plate and lever assembly and spaced apart from the first feature and positioned to provide inward tension to the pair of guidewire legs as the lever plate is rotated; and
a plurality of third features disposed symmetrically on the lever plate and positioned to provide outward tension to the guidewire legs as the lever plate is rotated.

2. A control handle for a steerable catheter as in claim 1, further comprising:
a cable having a pair of ends coupled to the guidewire legs adjacent the lever plate, the cable wrapped around the lever via the third features and affixed at the cable terminal points.

3. A control handle for a steerable catheter, comprising:
a housing having a length enclosing a hollow interior region, a top half, a bottom half, a front end and a back end;
a pair of circular sections symmetrically formed in a substantially central location along the length of both the top and bottom halves of the housing;
a circular plate formed within the circular section in the bottom half of the housing and having a smaller diameter than the circular section;
a saddle formed near the front of the bottom half of the housing for affixing a catheter tube received through an opening in the front housing;
a pair of vertical posts symmetrically formed and spaced apart laterally in the bottom half of the housing between the saddle and the circular plate;
a pair of slots portions symmetrically formed adjacent at least one side edge of the top and bottom halves;
a circular lever mechanism rotatably mounted on the circular plate;
at least one arm extending from the lever mechanism through the slot portions in the housing;
a pair of semicircular vertical walls symmetrically formed on opposite sides of the plate;
a pair of knob portions each formed at the front end of the respective vertical wall and each having a thickness that is more than the vertical wall;
a pair of semicircular tunnel portions each symmetrically formed in a central portion on the outside of a respective one of the vertical walls;
a first pair of cable openings symmetrically formed and spaced apart at the rear of the circular plate; and
a second pair of cable openings symmetrically formed and spaced apart at the rear of the circular plate in between the first pair of cable openings;
wherein a cable is routed around a first one of the pair of knob portions, through a first one of the pair of semicircular tunnels, then down through a first one of the first pair of cable openings, up through a first one of the second pair of cable openings, down again through a second one of the first pair of cable openings, up through a second one of the second pair of cable openings, then through a second one of the pair of semicircular tunnels, and around a second one of the pair of knobs; and
wherein a pair of ends of the cable are coupled to respective ones of a pair of guidewire legs extending from the catheter tube into an area between the pair of knob portions and the pair of vertical posts.
